(19) United States
(12) Patent Application Publication
(10) Pub. No.: US 2022/0054645 A1
(43) Pub. Date: Feb. 24, 2022

(54) Targeted Delivery of Therapeutic Molecules

(71) Applicant: Sirnaomics, Inc., Gatithersburg, MD (US)

(72) Inventors: Xiaoyong LU, Gaithersburg, MD (US); David M. EVANS, Gaithersburg, MD (US); Patrick Y. LU, Gaithersburg, MD (US); Alan LU, Gaithersburg, MD (US); Jon XU, Gaithersburg, MD (US); Peter Zhang, Suzhou (CN)

(73) Assignee: Peter ZHANG, Suzhou (CN)

(21) Appl. No.: 17/361,164

(22) Filed: Jun. 28, 2021

Related U.S. Application Data
(63) Continuation of application No. PCT/US2019/068205, filed on Dec. 22, 2019.
(60) Provisional application No. 62/786,213, filed on Dec. 28, 2018.

(51) Int. Cl. A61K 47/60; C12N 15/113; A61K 31/7115

(52) U.S. Cl. CPC A61K 47/60; A61K 31/7115; C12N 15/113

(57) ABSTRACT

The invention relates to the targeted delivery of therapeutic molecules to organs, tissues, and cells of humans and other mammals. The invention is directed to a chemical construct for delivering such therapeutic molecules and to methods of making and using the constructs.

### 1. CROSS-REFERENCE TO RELATED PATENT APPLICATIONS

This application is a continuation of International Application No. PCT/US2019/068205, filed on Dec. 22, 2019, which claims the benefit of and priority to U.S. Provisional Patent Application No. 62/786,213, filed Dec. 28, 2018, which is incorporated herein by reference in its entirety.

### 1.1 SEQUENCE LISTING

The instant application contains a Sequence Listing which has been submitted electronically in ASCII format and is hereby incorporated by reference in its entirety. Said ASCII copy, created on Feb. 14, 2020, is named 4690_0024 i_SL.txt and is 9,268 bytes in size.

### 2. FIELD OF INVENTION

The invention relates to the targeted delivery of therapeutic molecules to organs, tissues, and cells of humans and other mammals.

### 3. BACKGROUND OF THE INVENTION

Delivery of a therapeutic compound to a specific location (for instance, to desired organs, tissue, or cells) in the human body has many benefits, not only in enhancing the therapeutic efficacy but also improving the safety profile in terms of the dosage and clearance rate. Targeted delivery of therapeutics has attracted great interest in the drive to improve tumor treatment through increasing efficacy and reducing the side effects [1]. Additionally, efficient delivery of a therapeutic compound to a specific location in the body would minimize or avoid unintended side effects, such as the requirement for much higher doses to ensure delivery of an appropriate amount of material at the site of action with the prospect of producing unwanted side effects at these higher doses.

One of the methods that has been demonstrated to be effective in delivery of a therapeutic compound to the target location is by attaching the compound to a targeting ligand [2]. The ligand is selected to recognize and bind to its homing receptor (present on the exterior of the plasma membrane on the cell to be targeted) and, upon binding with the ligand attached to the therapeutic compound, translocates the compound into the cell to exert its therapeutic effect.

Among novel biologic drugs, including nucleotide-based medicines such as microRNA (miRNA), small interfering RNA (siRNA), and DNA vaccines, the potential of RNAi to silence any gene has made it an attractive therapeutic modality, since the discovery of a functional RNAi pathway in mammals has provided a powerful tool for reverse genetics as a method for selectively silencing a specific gene and decreasing production of a protein that is intrinsically responsible for the etiology of a disease. Recently, because of its sequence-specific post-transcriptional gene silencing ability, siRNA has become a promising novel therapeutic candidate for treating many diseases, such as cancer, infections, macular degeneration, cardiovascular disease, nervous system disorders, and other gene-related diseases. Due to their ability to knock down expression of any gene, siRNAs have been heralded as ideal candidates for treating a wide variety of diseases, including those with “undruggable” targets (i.e. those that are not available for access by monoclonal antibodies or do not have a clear site where a small molecule can block the activity of the protein).

Approaches to targeted delivery of siRNA in vivo have been challenging due to degradation of unmodified siRNAs by serum nucleases, rapid clearance, endosomal entrapment, and innate immunity simulation produced by the nanoparticle used for delivery [3].

### 4. BRIEF DESCRIPTION OF THE FIGURES

FIG. 1. Schematic illustration of general construct. The construct contains a linker-1 and linker-2, and a joint bridge which connects the ligand part and payload part, and a targeting ligand and a delivered payload. The payload is a therapeutic molecule.

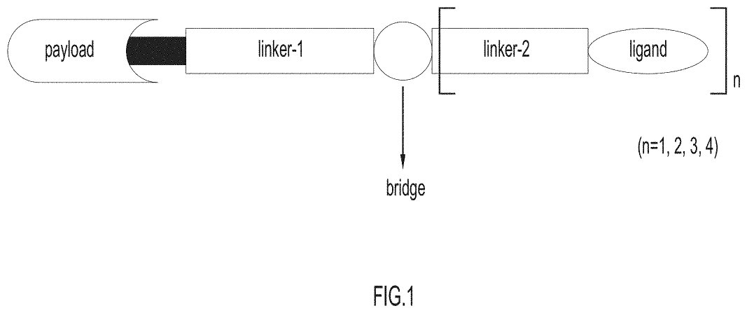

FIG. 2. Schematic illustration of ligand-conjugated siRNA. N-acetyl galactosamine (GalNAc) conjugated siRNA (TGFβ1 or cox2) consists of a 25-nucleotide sense strand and a 25-nucleotide antisense strand, in which the 3′ end of the antisense strand has a zero-nucleotide overhang. A GalNAc is conjugated to the 3′ or 5′ end of the sense strand, respectively. Three type of ligand are presented here respectively as trivalent-GalNAc conjugate, bivalent-GalNAc conjugate, monovalent-GalNAc conjugate, in which three, two and one ligand was attached in each case.

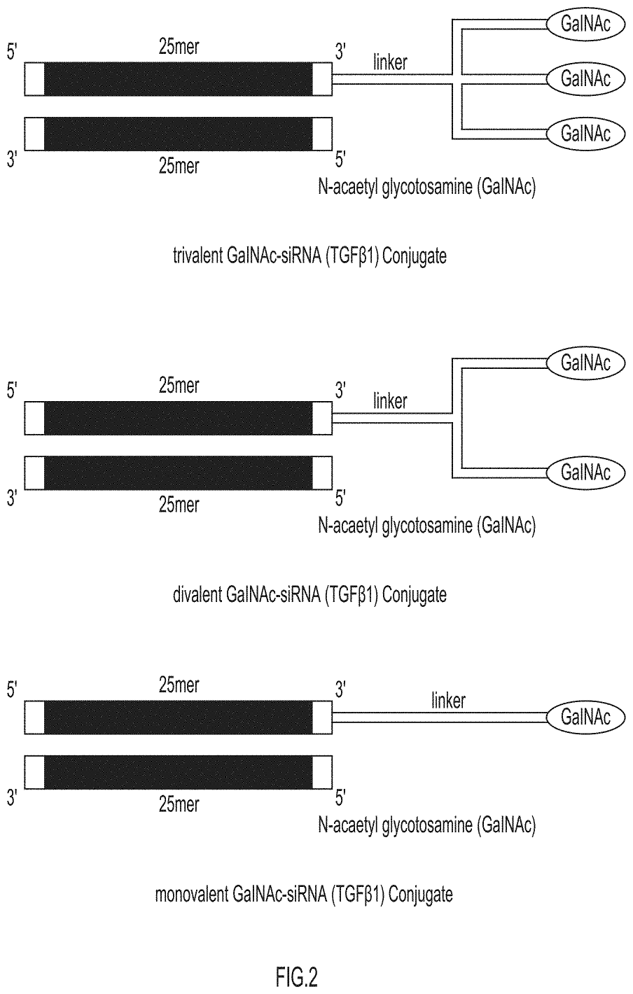

FIG. 3. Schematic illustration of alternative ligand-conjugated sense strand of siRNA. N-acetyl galactosamine (GalNAc) conjugated siRNA (TGFβ1 or Cox2) consists of a 25-nucleotide sense strand and a 25-nucleotide antisense strand, in which the 3′ end of the antisense strand has a zero-nucleotide overhang. A GalNAc is conjugated to the 3′ or 5′ end of the sense strand through an aliphatic chain, respectively. Three type of ligand are presented here respectively as trivalent-GalNAc conjugate (n=3), bivalent-GalNAc conjugate (n=2), monovalent-GalNAc conjugate (n=1), in which three, two and one ligand was attached in each case.

FIG. 4. Schematic illustration of alternative ligand-conjugated sense strand of siRNA. N-acetyl galactosamine (GalNAc) conjugated siRNA (TGFβ1) consists of a 25-nucleotide sense strand and a 25-nucleotide antisense strand, in which the 3′ end of the antisense strand has a zero-nucleotide overhang. The RNA is methylated as OMe (or partially modified) functional groups to improve the stability. A GalNAc is conjugated to the 5′ (or 3′) end of the sense strand through a phosphate, respectively. Three type of ligand are presented here respectively as trivalent-GalNAc conjugate (n=3), bivalent-GalNAc conjugate (n=2), monovalent-GalNAc conjugate (n=1), in which three, two and one ligand was attached in each case. The other 3′ (or 5′) end was conjugated with a cholesterol functional group to enhance the ability of membrane penetration. FIG. 4 discloses SEQ ID NO: 7.

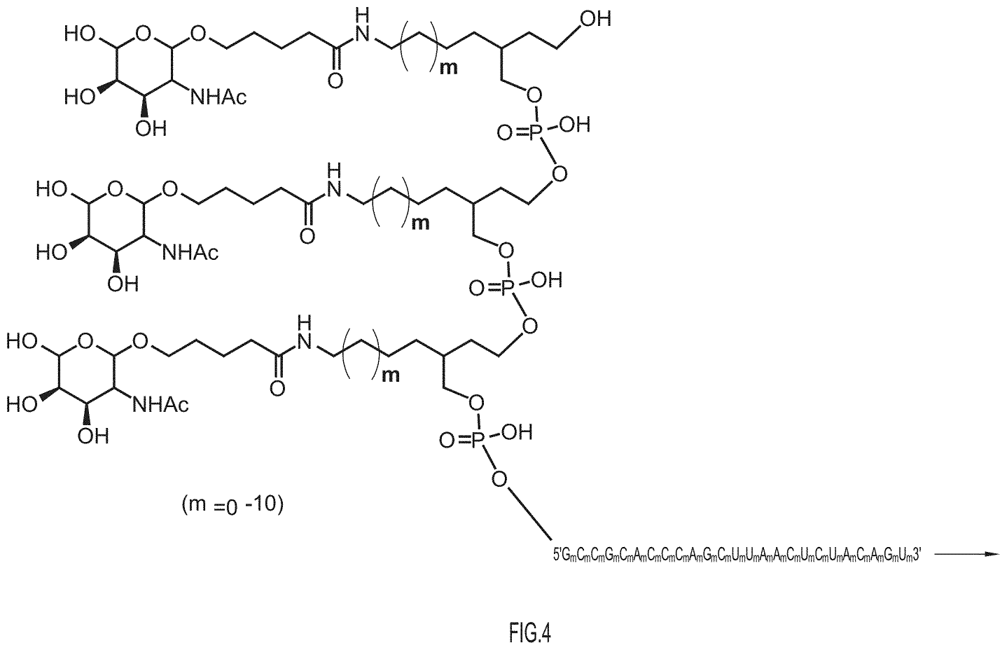

FIG. 5. Schematic illustration of alternative ligand-conjugated siRNA. N-acetyl galactosamine (GalNAc) conjugated siRNA (COX-2) consists of a 25-nucleotide sense strand and a 25-nucleotide antisense strand, in which the 3′ end of the antisense strand has a zero-nucleotide overhang. The RNA is methylated as OMe functional groups to improve the stability. A GalNAc is conjugated to the 5′ (or 3′) end of the sense strand through a phosphate, respectively. Three type of ligand are presented here respectively as trivalent-GalNAc conjugate (n=3), bivalent-GalNAc conjugate (n=2), monovalent-GalNAc conjugate (n=1), in which three, two and one ligand was attached in each case. The other 3′ (or 5′) end was conjugated with a cholesterol functional group to enhance the ability of membrane penetration. FIG. 5 discloses SEQ ID NO: 21.

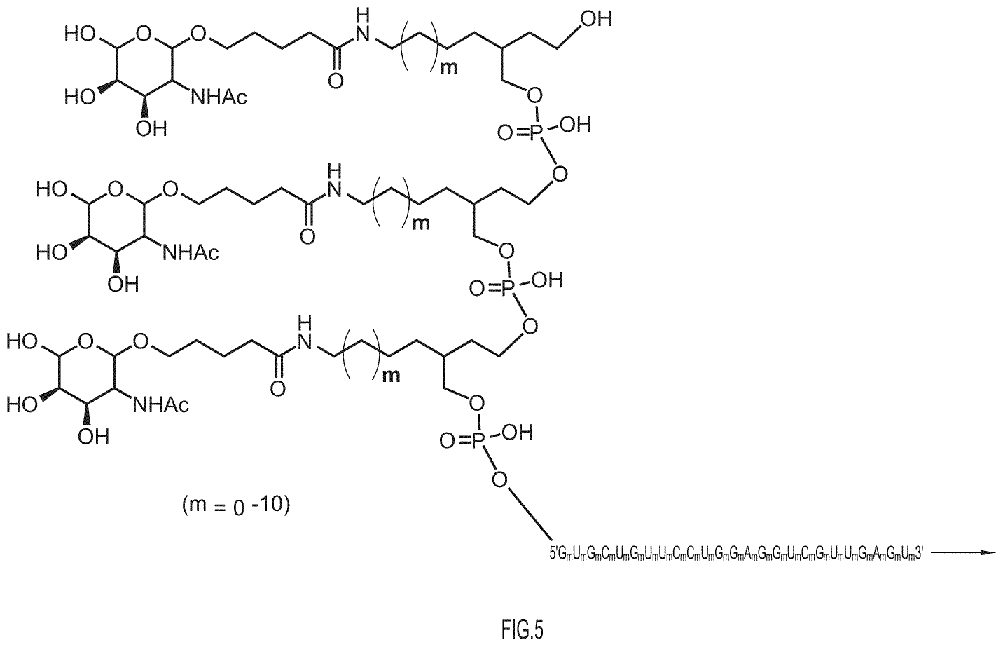

FIG. 6. Linkage type and synthetic route illustration between the siRNA sense strand and linker-ligand. The 3′ end of the sense strand was covalently modified by the chemical transformation through several synthetic steps to link the functionalized polyethylene glycol (Fun PEG) group. The 2′ position could be H or an OR group with a protection group such as TOM or TBDMS.

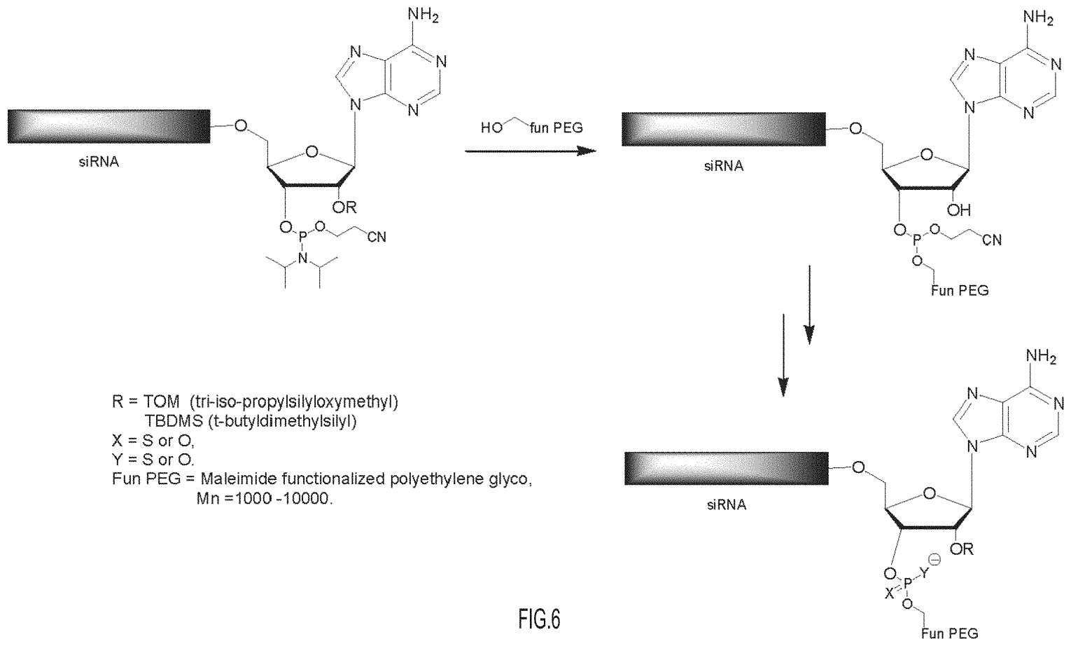

FIG. 7. The linker-1 design between the siRNA (TGFβ1) and ligand (ex. GalNAc). Two types of linker are described here for connecting the siRNA to the ligand. One is using a water soluble PEG with a terminal thiol that can act as the linker, the other one is poly (L-lactide) also with a terminal thiol to provide a site for linking to other moieties. Both of these products are readily available with various lengths and ready to conjugate to thiol groups using standard maleimide linkage chemistry [4].

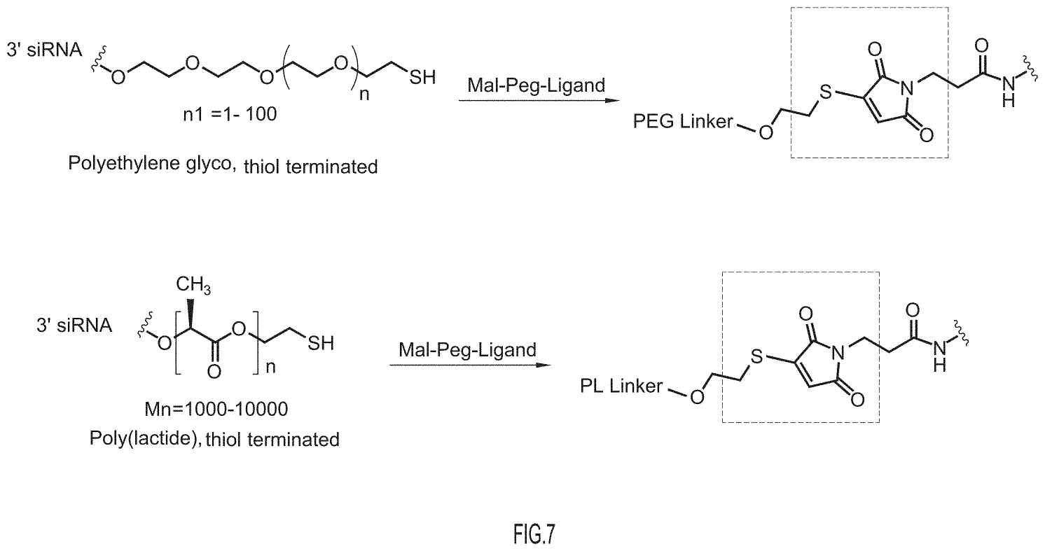

FIG. 8. The structure illustration of the ligand GalNAc molecule terminated by the maleimide functional group. A monovalent GalNAc molecule, a bivalent GalNAc molecule and a trivalent GalNAc molecule are shown. The three GalNAc ligands were linked to the tripodal linker through a triazole ring by using the “click” reaction between azide and alkyne [5]. The other end of the molecule was capped by the maleimide functional motif to allow further chemical modification.

FIG. 9. In vitro testing of GalNAc-siRNA in HepG2 cell line. GalNAc-TGFβ1 in FIG. 4 m=0 was used in this study on human hepatocellular carcinoma HepG2 cells viability. Effect of treatment with Cell Death siRNA was formulated with GalNAc-TGFβ1 (25 nM, 50 nM, 100 nM) along with control blank, none silence siRNA (NC, 100 nM), HKP (100 nM), liposome (25 nM, 50 nM and 100 nM, respectively), and liposome-GalNAc-TGFβ1 (25 nM, 50 nM, 100 nM, respectively). A mixture of GalNAc-TGFβ1 (25 nM, 50 nM, 100 nM, respectively), control blank, none silence siRNA (NC, 100 nM), HKP (100 nM), liposome (25 nM, 50 nM and 100 nM, respectively), liposome-GalNAc-TGFβ1 (25 nM, 50 nM, 100 nM, respectively) was incubated with cells in 100 μL OPTI-MEM medium. Transfection medium was replaced with 10% FBS/DMEM or EMEM in 6 h after. At 72 h post-transfection number of viable cells was assessed with Real-Time Quantitative Reverse Transcription QRT-PCR assay to quantify the TGF-β1 mRNA relative expression. Values derived from untreated cells (Blank) were set as 100%. NC-non-silencing siRNA.

FIG. 10. In vivo testing of GalNAc-siRNA in mouse model. The structure of GalNAc-TGFβ-1 in FIG. 4 (m=0) was used in this study. Dosages are siRNA per mouse and one injection was administered at 200 μg, 100 μg or 50 μg for GalNAc/siRNA-H (high concentration) to GalNAc/siRNA-L (low concentration), and PC (HKP/siRNA=4:1) is 40 μg. The mice were administrated by the designed dosage through tail-vein injection. After 24 hours drug administration, the right lobe of the livers in treated animals were collected and homogenized for RNA extraction. QRT-PCR was then performed to measure the degree of silencing of the relevant mRNA. Data shown are averages of 4 mice. *-P<0.05 V.S Blank, and **-P<0.01 V.S Blank. PC means positive control. Summary, in the positive control (PC), the HKP/siRNA was delivered to the whole liver, however, the GalNAc-H, -M, -L are specific to the liver hepatocytes only. So the overall mRNA expressed in liver is slightly higher in the GalNAc cases than the PC cases. And we also observed the dose dependent effect in GalNAc-H, -M, and -L. Overall, it strongly suggests that GalNAc has successfully delivered the siRNA and shows the silence effect.

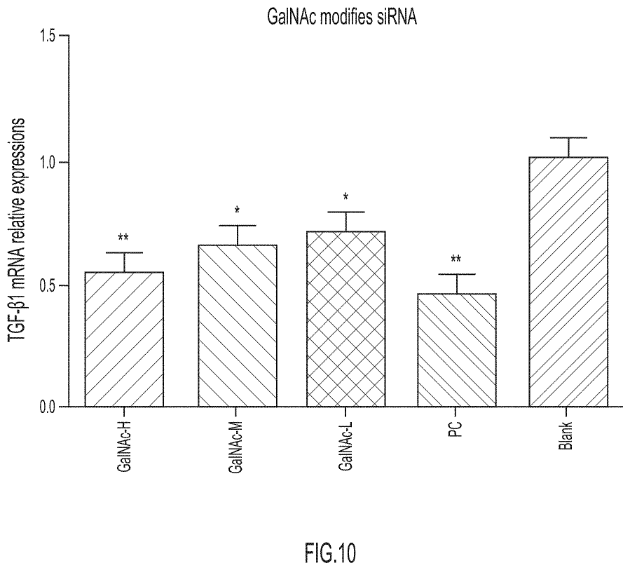

FIG. 11. Synthetic route for the monovalent GalNAc ligand. The synthesis of the monovalent GalNAc ligand is shown. The method employed several steps, mainly utilizing the “click” reaction between the two molecules and followed by amide formation between the NHS group and amine. Finally, it was terminated with the maleimide group.

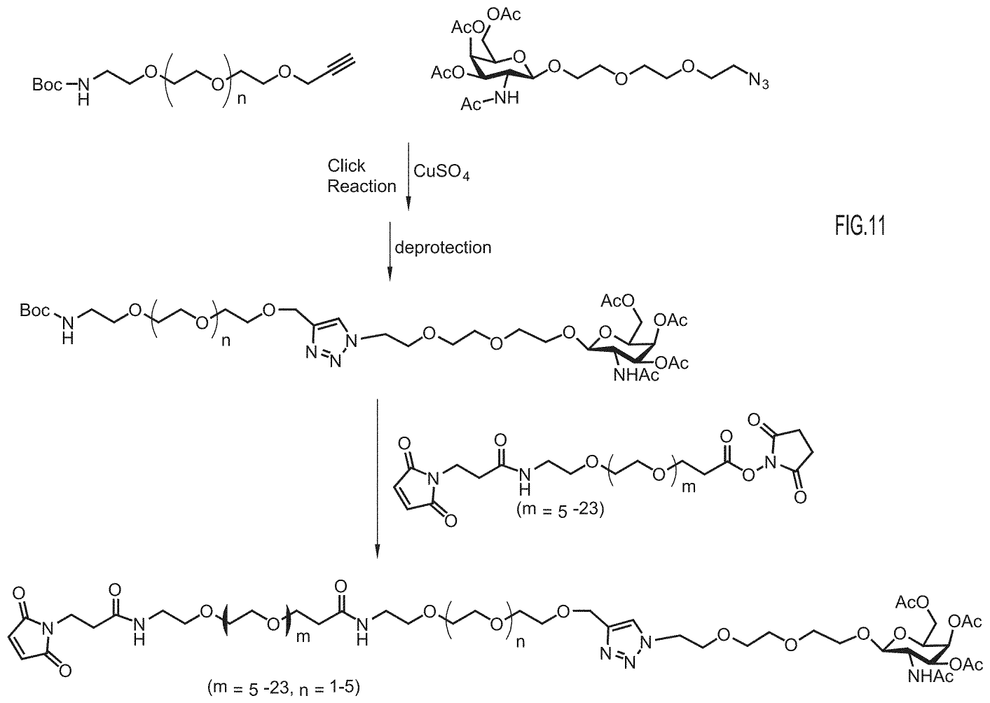

FIG. 12. Synthetic route of the divalent GalNAc ligand. The synthesis of the divalent GalNAc ligand is shown here through five steps, mainly by installation of the alkyne group, “click” reaction between azide-GalNAc, followed by the amide formation between the NHS group and amine. Finally, it was terminated with the maleimide group.

FIG. 13. Synthetic route of the trivalent GalNAc ligand. The synthetic route was very similar to that in FIG. 11 only the substrate was changed to tris(hydroxymethyl)-aminomethane.

FIG. 14. Synthetic route of the trivalent GalNAc ligand modified sense strand of siRNA (TGFβ1). GalNAc was conjugated on the 5′ end of the sense strand of siRNA consisting of a 25-nucleotide sense strand, in which the 3′ end of the sense strand can be further modified by other functional groups. FIG. 14 discloses SEQ ID NOS 7 and 7, respectively, in order of appearance.

FIG. 15. Synthetic route for the trivalent GalNAc ligand modified sense strand of siRNA (COX-2). GalNAc was conjugated to the 5′ end of the sense strand of siRNA consisting of a 25-nucleotide sense strand, in which the 3′ end of the sense strand can be further modified by other functional groups. FIG. 15 discloses SEQ ID NOS 21 and 21, respectively, in order of appearance.

FIG. 16. Preparation of the trivalent GalNAc ligand modified siRNA. The siRNA duplex chemically modified with a thiol containing linker at 3′ (or 5′) end of the sense strand or antisense strand as illustrated in the FIG. 16. This construct was then conjugated with the pre-prepared trivalent GalNAc ligand through the thiol/maleimide chemistry in 1.2 to 1 molar ratio in the formation of a thiol-carbon bond in a pH 7.4-9.0 buffer. The resulted GalNAc conjugated siRNA can be used directly as the buffer from for in vitro study, or dialysis by membrane to remove the salt and lyophilize into to solid form.

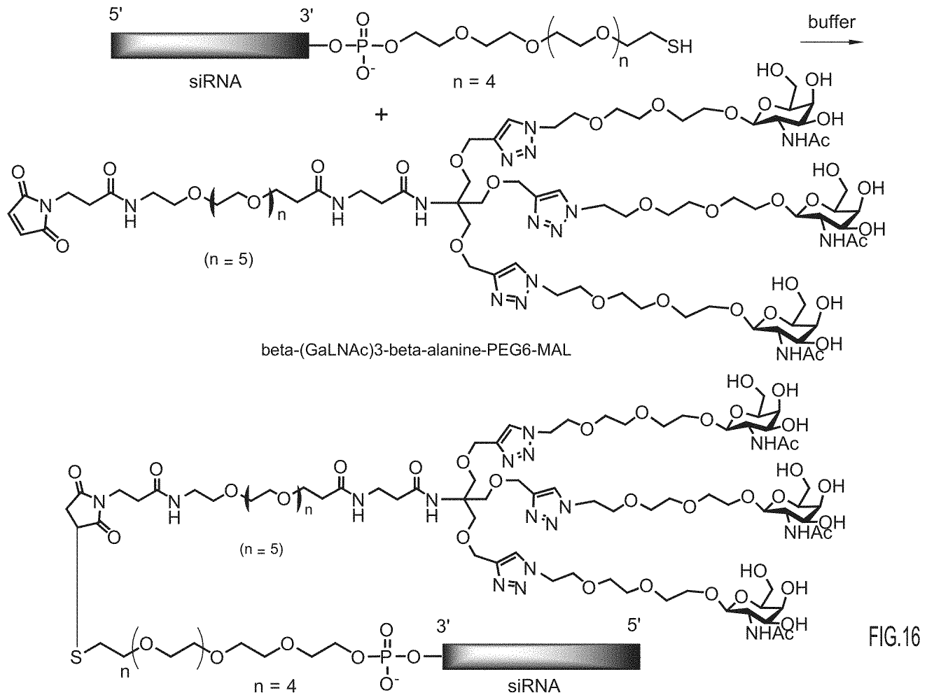

FIG. 17. 1H NMR spectrum (D₂O, 400 MHz) of the trivalent GalNAc-PEG6-Mal terminated with a maleimide.

FIG. 18. Mass spectrum (ESI-MS, positive) of the trivalent GalNAc-PEG6-Mal terminated with a maleimide.

FIG. 19. HPLC spectrum of the trivalent GalNAc-PEG6-Mal terminated with a maleimide.

### 5. DESCRIPTION OF THE INVENTION

The invention is directed to a chemical construct for delivering therapeutic molecules to mammalian cells, preferably human cells, and most preferably human cells in the human body. The construct is represented by the formula (I):

A-B—[—C-D]ₙ (I)

where A is a first linker (linker 1), B is a bridge, C is a second linker (linker 2), D is a targeting ligand, and n is an integer from 1-4. Linker 1 and linker 2 can be the same or different. In one embodiment, n=1. In another embodiment, n=3.

Linkers are selected to be suitable for the usage in the constructs disclosed herein, including a water-soluble, flexible polyethylene glycol (PEG) which is sufficiently stable and limits the potential interaction between one or more targeting moiety(s). In addition, PEG has been validated to be safe and compatible for therapeutic purposes from clinical studies. In some embodiments, the linker can be poly(L-lactide) with a selected range of the molecular weight for suitable delivery of the targeting compound with a biodegradable nature in the ester bond. The linker reactive connection moiety includes, but is not limited to, a thiol-maleimide linkage, triazole of alkyne-azide linkage, and amide of an amine-NHS linkage.

In one embodiment, linker 1 is a linear polyethylene glycol as shown in the first structure below, where n1 is an integer between 1-50, or linker 1 is a poly(L-lactide) as shown in the second structure below, where n2 is an integer from 1-70, and where Z (shown in both structures below) is a functional group, such as thiol or carboxylic acid, which will react with a maleimide or an amine to conjugate covalently with the bridge.

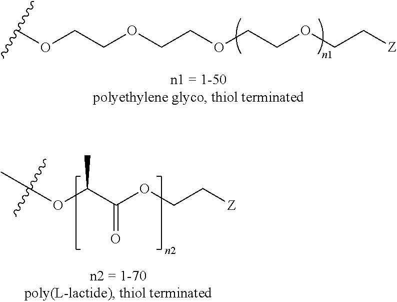

In another embodiment, linker 1 has a sub-chemical group Z comprising a thiol-maleimide bond as shown:

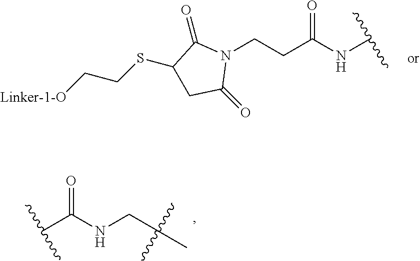

or any other pair of the conjugation chemistries shown below, which also can be used in the linker 2 conjugation with the bridge:

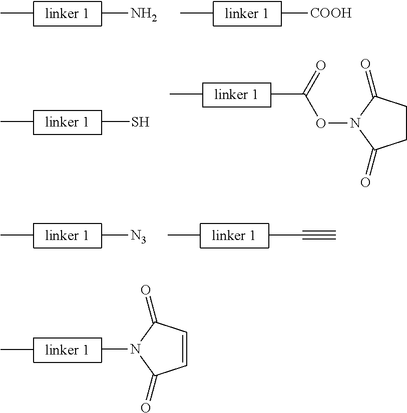

In one aspect of this embodiment, Z is a docking site that chemically attaches linker 1 and the bridge.

In one embodiment, linker 2 is a tri-, tetra-, or penta-ethylene glycol. In one aspect of this embodiment, a chemical structure comprising linker 2 and 1-3 of the targeting ligands is attached to the bridge, where the chemical structure comprises one of the following structures:

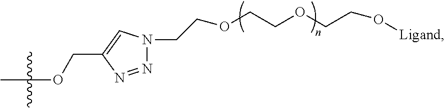

where n is 1, 2, or 3 and is connected to the bridge through a 1, 5-triazol ring with an OCH₂ unit; or

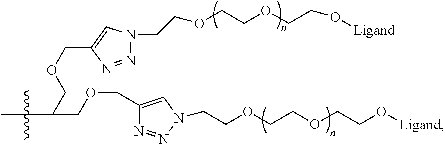

where n is 1, 2, or 3 and is connected to the bridge through a 1, 5-triazol ring with an CH₂OCH₂ unit; or

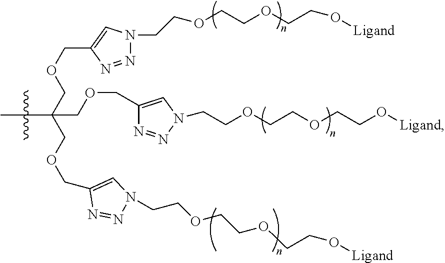

where n is 1, 2, or 3 and is connected to the bridge through a 1, 5-triazol ring with an CH₂OCH₂ unit.

In one embodiment, the bridge is a chemical structure connecting linker 1 and linker 2, where the chemical structure is a linear structure —CH₂OCH₂—, a single-branched structure

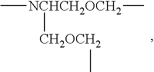

or double-branched tripodal structure

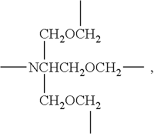

and where the bridge is directly connected to the triazole ring of linker 2.

In another embodiment, the bridge is a linear structure with the formula

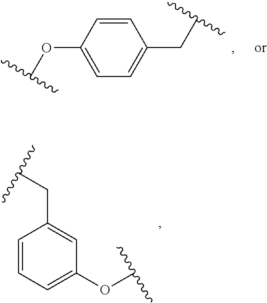 [SMILES: *Cc1ccc(O*)cc1 ; *Cc1cccc(O*)c1]

which allows only one chemical construct comprising linker 2 and a targeting ligand to be conjugated at the para-position, or a branched structure with the formula

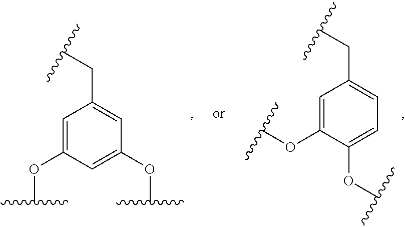 [SMILES: *Cc1cc(O*)cc(O*)c1 ; *Cc1ccc(O*)c(O*)c1]

which allows two chemical constructs comprising linker 2 and a targeting ligand to be conjugated at the two meta-positions, or a tripodal structure with the formula

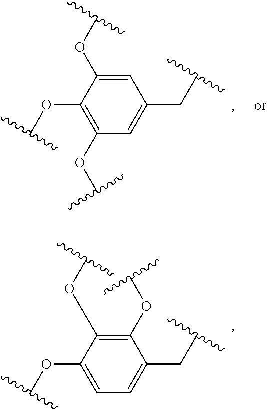 [SMILES: *Cc1cc(O*)c(O*)c(O*)c1 ; *Cc1ccc(O*)c(O*)c1O*]

which allows three chemical constructs comprising linker 2 and a targeting ligand to be conjugated at the two meta- and one para-positions.

In one embodiment of the chemical construct, linker 1 is a linear aliphatic chain conjugated by an internal amide bond and linker 2 and the bridge have been replaced with a phosphate linkage, as shown in the following structure:

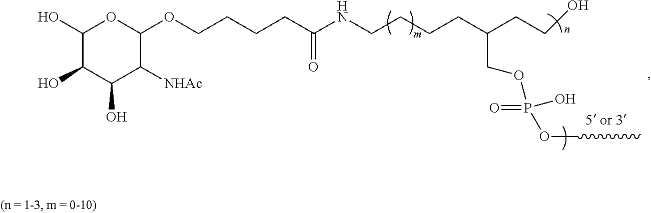

where m is 0-10 and n is 1-3.

In one embodiment of the construct, the targeting ligand is N-acetyl-galactosamine (GalNAc), galactose, galactosamine, N-formal-galactosoamine, N-propionyl-galactosamine, or N-butanoylgalactosamine. In one aspect of this embodiment, the targeting ligand is N-acetyl-galactosamine (GalNAc).

The construct (I) can be directly coupled to a therapeutic molecule through linker 1, forming a new construct with the formula (II):

TM-A-B—[—C-D]ₙ (II)

where TM is a therapeutic molecule, A is a first linker (linker 1), B is a bridge, C is a second linker (linker 2), D is a targeting ligand, and n is an integer from 1-4. As used herein, a therapeutic molecule is a molecule that has a therapeutic effect in the human body. Such therapeutic molecules include expression-inhibiting oligonucleotides, therapeutic peptides, antibodies with therapeutic efficacy, and small molecules with therapeutic efficacy.

In one embodiment of this second construct (II), the expression-inhibiting oligonucleotide is an RNAi, an anti-sense RNA, or a cDNA. In one aspect of this embodiment, the RNAi is an siRNA or a miRNA. In a further aspect of this embodiment, the RNAi is an siRNA.

In another embodiment, the second construct is represented by the formula (III):

O-A-B—[—C-D]ₙ (III)

where O is an oligonucleotide, A is a first linker (linker 1), B is a bridge, C is a second linker (linker 2), D is a targeting ligand, and n is an integer from 1-4. Such oligonucleotides include an RNAi, an anti-sense RNA, or a cDNA. A, B, C, and D are as described above. In one aspect of this embodiment, the oligonucleotide is double stranded. In another aspect, the oligonucleotide is single-stranded. In one aspect of this embodiment, the oligonucleotide is partially chemically modified.

In one aspect of this embodiment, the RNAi is an siRNA or a miRNA. In a further aspect of this embodiment, the RNAi is an siRNA. In another aspect, the RNAi is double-stranded and covalently bonded to linker 1 through a phosphate, phosphorothioate, or phosphonate group at 3′ terminal end of the sense strand of the RNAi.

In still another aspect, the oligonucleotide is an siRNA. Preferably, the siRNA is between 10-27 nucleotides in length. Most preferably, it is between 19-25 nucleotides in length. Preferably, the targeting ligand is GalNAc.

In another aspect, the first construct (I) is covalently connected to an siRNA molecule at the 3′ position or the 5′ position through linker 1 as shown below, x=O or S, y=O or S:

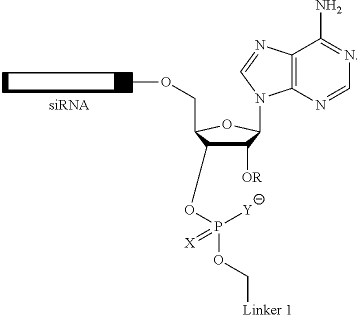

As used herein, an siRNA molecule is a duplex oligonucleotide, that is a short, double-stranded polynucleotide, that interferes with the expression of a gene in a cell, after the molecule is introduced into the cell. For example, it targets and binds to a complementary nucleotide sequence in a single stranded target RNA molecule. SiRNA molecules are chemically synthesized or otherwise constructed by techniques known to those skilled in the art. Such techniques are described in U.S. Pat. Nos. 5,898,031, 6,107,094, 6,506,559, 7,056,704 and in European Pat. Nos. 1214945 and 1230375, which are incorporated herein by reference in their entireties. By convention in the field, when an siRNA molecule is identified by a single, particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

In a particular aspect, the siRNA is an anti-TGFbeta 1 siRNA. As used herein, an anti-TGFbeta 1 siRNA is an siRNA molecule that reduces or prevents the expression of the gene in a human or other mammalian cell that codes for the synthesis of TGFbeta 1 protein.

In another particular aspect, the siRNA is an anti-Cox2 siRNA. As used herein, an anti-Cox2 siRNA is an siRNA molecule that reduces or prevents the expression of the gene in a human or other mammalian cell that codes for the synthesis of Cox2 protein.

In another particular aspect, the oligonucleotide of siRNA is fully or partially chemically modified at 2′ position to improve the stability.

TABLE 1
Potent siRNAs targeting TGF-beta 1 and Cox2:

| | | |
|---|---|---|
| hmTFβ1a: | Sense: | 5′ r(GGAUCCACGAGCCCAAGGGCUACCA)-3′ (SEQ ID NO: 1) |
| | Anti-sense: | 5′-r(UGGUAGCCCUUGGGCUCGUGGAUCC)-3′ (SEQ ID NO: 2) |
| hmTFβ1b: | Sense: | 5′-r(CCCAAGGGCUACCAUGCCAACUUCU)-3′ (SEQ ID NO: 3) |
| | Anti-sense: | 5′r(AGAAGUUGGCAUGGUAGCCCUUGGG)-3′ (SEQ ID NO: 4) |
| hmTFβ1c: | Sense: | 5′r(GAGCCCAAGGGCUACCAUGCCAACU)-3′ (SEQ ID NO: 5) |
| | Anti-sense: | 5′-r(AGUUGGCAUGGUAGCCCUUGGGCUC)-3′ (SEQ ID NO: 6) |
| hmTFb1d: | Sense: | 5′ (GₘCₘCₘGₘCₘAₘCₘCₘCₘAₘGₘCₘUₘUₘAₘAₘCₘUₘCₘUₘAₘCₘAₘGₘUₘ)3′ |
| | | (SEQ ID NO: 7) |
| | Anti-sense: | 5′ ACUGUAGAGUUAAGCUGGGUGCGGC 3′ (SEQ ID NO: 8) |
| hmTFb1e: | Sense: | 5′ (GₘCₘCₘGₘCₘAₘCₘCₘCₘAₘGₘCₘU_{F}U_{F}A_{F}A_{F}CₘUₘCₘUₘAₘCₘAₘGₘUₘ)3′ |
| | | (SEQ ID NO: 9) |
| | Anti-sense: | 5′ A_{F}CU_{F}GU_{F}AG_{F}AG_{F}UU_{F}AA_{F}GC_{F}UG_{F}GG_{F}UG_{F}CG_{F}GC_{F} 3′ (SEQ ID NO: 10) |
| hmTFb1f: | Sense: | 5 (GₘCₘCₘGₘCₘAₘCₘCₘCₘAₘGₘCₘU_{F}U_{F}A_{F}A_{F}CₘUₘCₘUₘAₘCₘAₘGₘUₘ)3′ |
| | | (SEQ ID NO: 9) |
| | Anti-sense: | 5′ ACUGUAGAGUUAAGCUGGGUGCGGC 3′ (SEQ ID NO: 8) |
| hmTF25d: | Sense: | 5′-r(GAUCCACGAGCCCAAGGGCUACCAU)-3′ (SEQ ID NO: 11) |
| | Anti-sense: | 5′ r(AUGGUAGCCCUUGGGCUCGUGGAUC)-3′ (SEQ ID NO: 12) |
| hmTF25e: | Sense: | 5′r(CACGAGCCCAAGGGCUACCAUGCCA)-3′ (SEQ ID NO: 13) |
| | Anti-sense: | 5′-r(UGGCAUGGUAGCCCUUGGGCUCGUG)-3′ (SEQ ID NO: 14) |
| hmTF25f: | Sense: | 5′-r(GAGGUCACCCGCGUGCUAAUGGUGG)-3′ (SEQ ID NO: 15) |
| | Anti-sense: | 5′ r(CCACCAUUAGCACGCGGGUGACCUC)-3′ (SEQ ID NO: 16) |
| hmTF25g: | Sense: | 5′ r(GUACAACAGCACCCGCGACCGGGUG)-3′ (SEQ ID NO: 17) |
| | Anti-sense: | 5′-r(CACCCGGUCGCGGGUGCUCUUCUAC)-3′ (SEQ ID NO: 18) |
| hmTF25h: | Sense: | 5′-r(GUGGAUCCACGAGCCCAAGGGCUAC)-3′ (SEQ ID NO: 19) |
| | Anti-sense: | 5′ r(GUAGCCCUUGGGCUCGUGGAUCCAG)-3′ (SEQ ID NO: 20) |
| COX2: | Sense: | 5′ (GₘUₘGₘCₘUₘGₘUₘUₘCₘCₘUₘGₘGₘAₘGₘGₘUₘCₘGₘUₘUₘGₘAₘGₘUₘ)3′ |
| | | (SEQ ID NO: 21) |
| | Anti-sense: | 5′ ACUCAACGACCUCCAGGAACAGCAC 3′ (SEQ ID NO: 22) |
| COX2a | Sense: | 5′ (GₘUₘGₘCₘUₘGₘUₘUₘCₘCₘUₘGₘG_{F}A_{F}G_{F}G_{F}UₘCₘGₘUₘUₘGₘAₘGₘUₘ)3′ |
| | | (SEQ ID NO: 23) |
| | Anti-sense: | 5′ ACUCAACGACCUCCAGGAACAGCAC 3′ (SEQ ID NO: 22) |
| COX2b | Sense: | 5′ (GₘUₘGₘCₘUₘGₘUₘUₘCₘCₘUₘGₘG_{F}A_{F}G_{F}G_{F}UₘCₘGₘUₘUₘGₘAₘGₘUₘ)3′ |
| | | (SEQ ID NO: 23) |
| | Anti-sense: | 5′ A_{F}CU_{F}CA_{F}AC_{F}GA_{F}CC_{F}UC_{F}CA_{F}GG_{F}AA_{F}CA_{F}GC_{F}AC_{F} 3′ (SEQ ID NO: 24) |

Note:
Xₘ, nucleotide 2′ OMe modification. X_{F}, nucleotide 2′ F modification.

Certain anti-TGFβ1 and anti-Cox-2 siRNA molecules are described in U.S. Pat. No. 9,642,873 B2, dated May 9, 2017, and U.S. Reissued Pat. RE46,873 E, dated May 29, 2018, the disclosures of which are incorporated by reference herein in their entireties.

In one embodiment of the second construct (II), the therapeutic molecule is a therapeutic peptide. Such therapeutic peptides include cyclic(c) RGD, APRPG (SEQ ID NO: 25), NGR, F3 peptide, CGKRK (SEQ ID NO: 26), LyP-1, iRGD (CRGDRCPDC) (SEQ ID NO: 27), iNGR, T7 peptide (HAIYPRH) (SEQ ID NO: 28), MMP2-cleavable octapeptide (GPLGIAGQ) (SEQ ID NO: 29), CP15 (VHLGYAT) (SEQ ID NO: 30), FSH (FSH-β, 33-53 amino acids, YTRDLVKDPARPKIQKTCTF) (SEQ ID NO: 31), LHRH (QHTSYkcLRP), gastrin-releasing peptides (GRPs) (CGGNHWAVGHLM) (SEQ ID NO: 32), RVG (YTWMPENPRPGTPCDIFTNSRGKRASNG) (SEQ ID NO: 33), FMDV20 peptide sequence (NAVPNLRGDLQVLAQKVART) (SEQ ID NO: 34), or GLP.

In another embodiment of the second construct (II), the therapeutic molecule is an antibody for therapeutic use. Such therapeutic antibodies include IgM, IgD, IgG, IgA, IgE, or antibody fragments F(ab′)2, Fab, Fab′, or Fv.

In still another embodiment of the second construct (II), the therapeutic molecule is a small molecule for therapeutic use. Such therapeutic small molecules include gemcitabine, folic acid, cisplatin, oxaliplatin, carboplatin, doxorubicin, or paclitaxel.

The constructs of the invention can be synthesized by persons skilled in the art, given the structures and teachings disclosed herein. For example, where the therapeutic molecule in the second construct is an siRNA molecule, the construct can be synthesized by the following steps:

1) conjugating the sense strand of the siRNA molecule to a functionalized linker 1 at the 5′ or 3′ site of the siRNA molecule through formation of a phosphate bond;

2) connecting one to three targeting ligand-linker 2 molecules to the tripodal, dipodal, or linear bridge site; at the other end of the bridge is a preinstalled short PEG group terminated with a maleimide group, which is used to conjugate linker 1 to the bridge;

3) conjugating the siRNA-linker-1 construct with the linker-2-targeting ligand construct through a thiol/maleimide reaction to provide a construct of the sense strand of the siRNA molecule with the one to three targeting ligand molecules; and

4) mixing the sense strand-targeting ligand construct with the antisense strand of the siRNA molecule to form the duplex siRNA with the one to three targeting ligands.

Where the therapeutic molecule in the second construct is an antibody or a peptide, the construct can be synthesized by the following steps:

1) conjugating the antibody (or peptide) molecule to a functionalized linker-1 (such as azido, maleimide, amine) at the alkyne, thiol, NHS functionalized site of the antibody (or peptide) molecule through formation of a triazole ring, a thiol-carbon bond, or an amide bond;

2) connecting one, (two, or three) of the targeting ligand-linker 2 constructs to the center linear linker, (dipodal, or tripodal bridge) site; at the other end of the bridge is conjugated with short PEG group with a maleimide functional group at the end, which is used to conjugate linker 1 to the bridge; (and

3) conjugating the antibody (or peptide)-linker-1 construct with the linker-2-targeting ligand construct through a thiol/maleimide reaction to provide a construct of the antibody (or peptide) with targeting ligand.

Where the therapeutic molecule in the second construct is an siRNA molecule and the targeting ligand is GalNAc, the construct can be synthesized by the following steps:

1) constructing a GalNAc-linker-2-bridge by connecting one to three GalNAc-linker 2 molecules to the tripodal, dipodal, or linear bridge site; at the other end of the bridge is a preinstalled short PEG group terminated with a maleimide group, which is used to conjugate linker 1 to the bridge;

2) reacting linker 1, such as PEG or poly(L-lactide) containing a thiol group moiety, with the terminal maleimide on the bridge-linker 2-GalNAc moiety to form a S—C covalent bond;

3) conjugating the ligand-linker 2-linker 1 construct to the 5′ or 3′ end of the sense strand of the siRNA molecule through a phosphate bond between the phosphonamidite group and the hydroxyl group; and

4) mixing the sense strand-GalNAc construct with the antisense strand of the siRNA molecule to form the siRNA duplex with the one to three GalNAc ligands.

The construct (I) of the invention can be indirectly coupled to a therapeutic molecule through a delivery agent such as cell penetration peptide and/or endosomal releasing agent. The construct (I) is first coupled with a short functional peptide (3-20 amino acid, such as cell endosomal releasing peptide HHHK (SEQ ID NO: 35), HHHHK (SEQ ID NO: 36), (HHHK)n, n=1-5, etc.) (SEQ ID NO: 37). The therapeutic molecule (such as antisense oligonucleotide, siRNA, DNA, aptamer, peptides, small molecule drugs, etc.) is then conjugated with the functional peptide.

The invention also includes pharmaceutical compositions. In one embodiment, the composition comprises the first construct (I) described above in a pharmaceutically acceptable carrier. In another embodiment, the composition comprises the second construct (II) or third construct (III) described above in a pharmaceutically acceptable carrier. In one aspect of both embodiments, the pharmaceutically acceptable carrier comprises water and one or more of the following salts or buffers: potassium phosphate monobasic anhydrous NF, sodium chloride USP, sodium phosphate dibasic heptahydrate USP, and Phosphate Buffered Saline (PBS).

The constructs and pharmaceutical compositions of the invention are useful for delivering therapeutic molecules to human cells, whether in vitro or in vivo. As disclosed above, such therapeutic molecules include expression-inhibiting oligonucleotides, therapeutic peptides, therapeutically efficacious antibodies, and therapeutically efficacious small molecules.

When used in vivo, the constructs and pharmaceutical compositions are used to treat human disease. In one embodiment, a therapeutically effective amount of a pharmaceutical composition of the invention is delivered to a human with a disease in need of treatment.

One category of such a disease is human cancer. Such cancers include liver cancer, cholangiocarcinoma (CCA), colon cancer, pancreatic cancer, lung cancer, bladder cancer, ovarian cancer, head and neck cancer, esophageal cancer, brain cancer, and skin cancers, including melanoma and non-melanoma skin cancers. In one aspect of this embodiment, the cancer is liver cancer, colon cancer, or pancreatic cancer.

In a particular aspect, the cancer is liver cancer. The liver cancer can be a primary liver cancer or a cancer that has metastasized to the liver from another tissue in the person's body. Primary liver cancer includes a hepatocellular carcinoma or a hepatoblastoma. Metastasized cancer includes colon cancer and pancreatic cancer.

Other human diseases are treatable with the constructs and pharmaceutical compositions of the invention. Such diseases include hepatitis, fibrosis, and primary sclerosing cholangitis (PSC). A therapeutically effective amount of the pharmaceutical composition of the invention is administered to a patient in need of treatment.

The constructs and pharmaceutical compositions of the invention are also useful in gene therapy. A therapeutically effective amount of the pharmaceutical composition of the invention is administered to a human or other mammal in need of such therapy. Other mammals include laboratory animals, such as rodents, guinea pigs, and ferrets, pets, and nonhuman primates.

The following examples illustrate certain aspects of the invention and should not be construed as limiting the scope thereof.

### 6. EXAMPLES

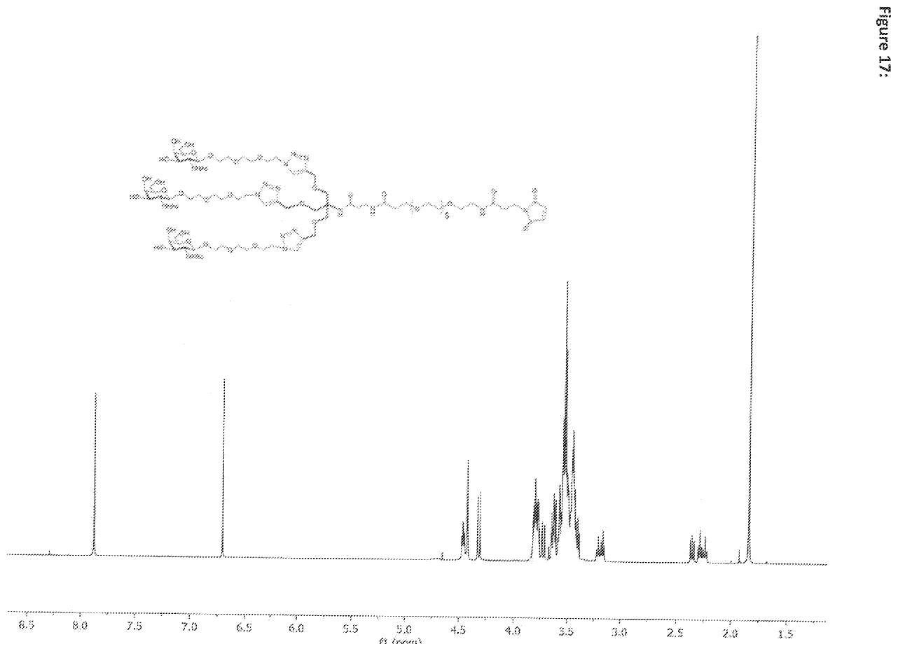

### Example 1. An ¹H NMR spectrum (D₂O, 400 MHz) of the trivalent GalNAc-PEG6-Mal terminated with a maleimide is shown in FIG. 17

The GalNAc was linked to the tripodal center through a triethylene glycol by triazole ring by “click” reaction. A hexa-PEG was used in the other end to connect with the maleimide.

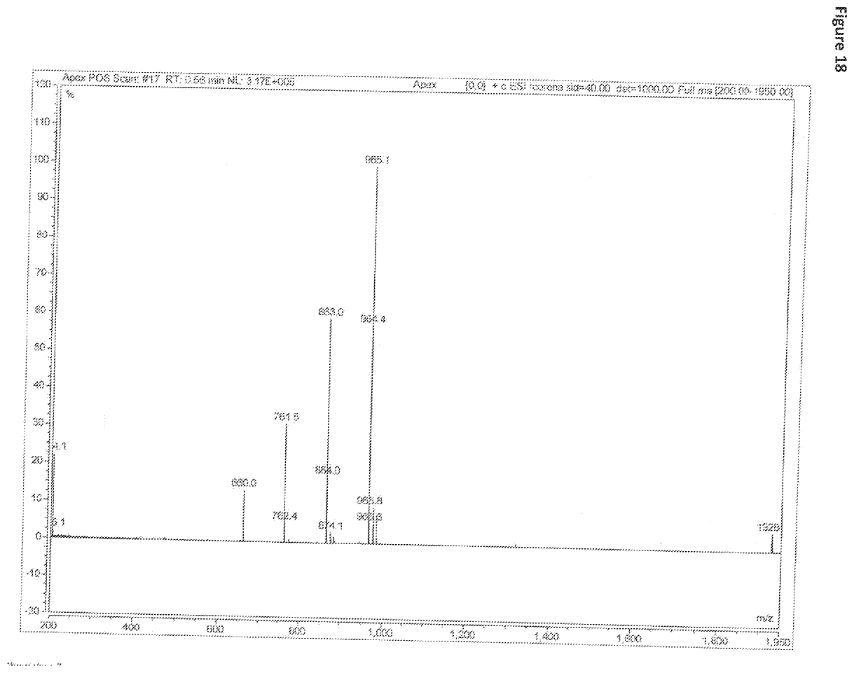

### Example 2. A Mass Spectrum (ESI-MS, Positive) of the Trivalent GalNAc-PEG6-Mal Terminated with a Maleimide is Shown in FIG. 18

Molecular ion was found as [M+H]⁺=1928.1, calculated as 1928.

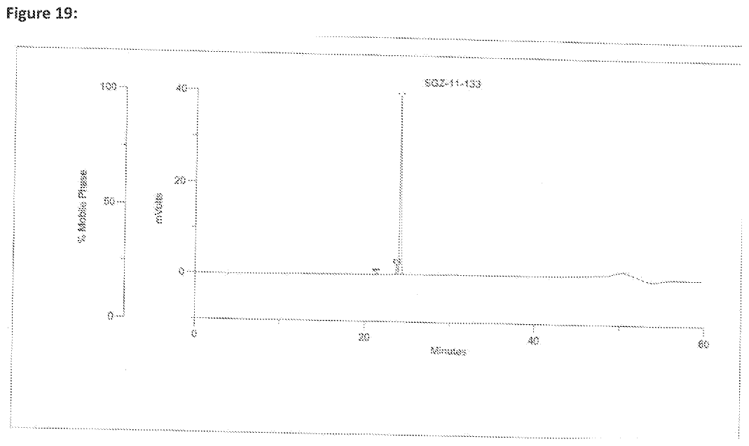

### Example 3. An HPLC Spectrum (C18 Column, 0.1% TFA Water/0.1% TFA Acetonitrile Gradient) of the Trivalent GalNAc-PEG6-Mal Terminated with a Maleimide is Shown in FIG. 19

### Example 4. Sequence and Structure of the TGFβ1 and COX-2

The sequence of the sense strand and antisense strand are shown below. Modifications are made at all nucleotides within the sense strand which is fully methylated. The 5′ end of the sense strand was conjugated by GalNAc ligand through a linker, 3′ end of the sense strand was chemically modified by a cholesterol to improve the ability of membrane penetration.

| Name | Sequence (5′ or 3′) | Modified | MS | HPLC |
|---|---|---|---|---|
| TGFβ1-GalNAc | Sense | 5′-GalNAc, 3′- | 10386 | >92% |
| | 5′GₘCₘCₘGₘCₘAₘCₘCₘCₘAₘGₘCₘUₘUₘAₘAₘ | cholesterol, fully OMe | | |
| | CₘUₘCₘUₘAₘCₘAₘGₘUₘ3′ (SEQ ID NO: 7) | methylation | | |
| | Antisense | No modification | 8093 | |
| | ACUGUAGAGUUAAGCUGGGUGCGGC | | | |
| | (SEQ ID NO: 8) | | | |
| COX2-GalNAc | Sense | 5′-GalNAc, 3′- | 10714 | >92% |
| | 5′GₘUₘGₘCₘUₘGₘUₘUₘCₘCₘUₘGₘGₘAₘGₘG | cholesterol, fully OMe | | |
| | ₘUₘCₘGₘUₘUₘGₘAₘGₘUₘ3′ (SEQ ID NO: 21) | methylation | | |
| | Antisense | No modification | 7945 | |
| | ACUCAACGACCUCCAGGAACAGCAC (SEQ ID | | | |
| | NO: 22) | | | |

### Example 5. In Vitro Testing of GalNAc-siRNA in HepG2 Cell Line

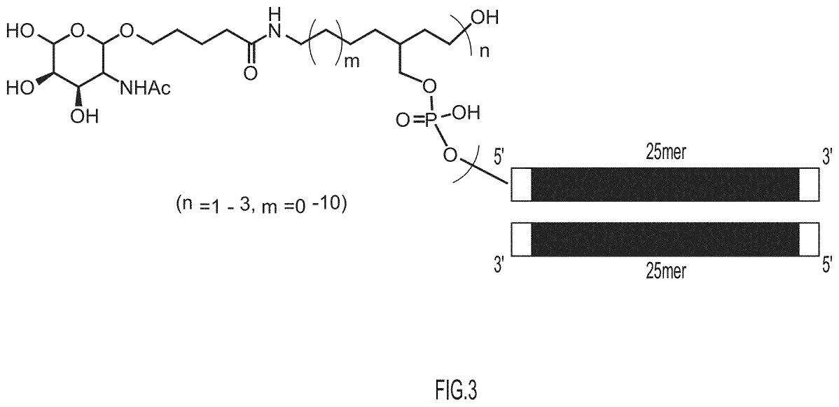

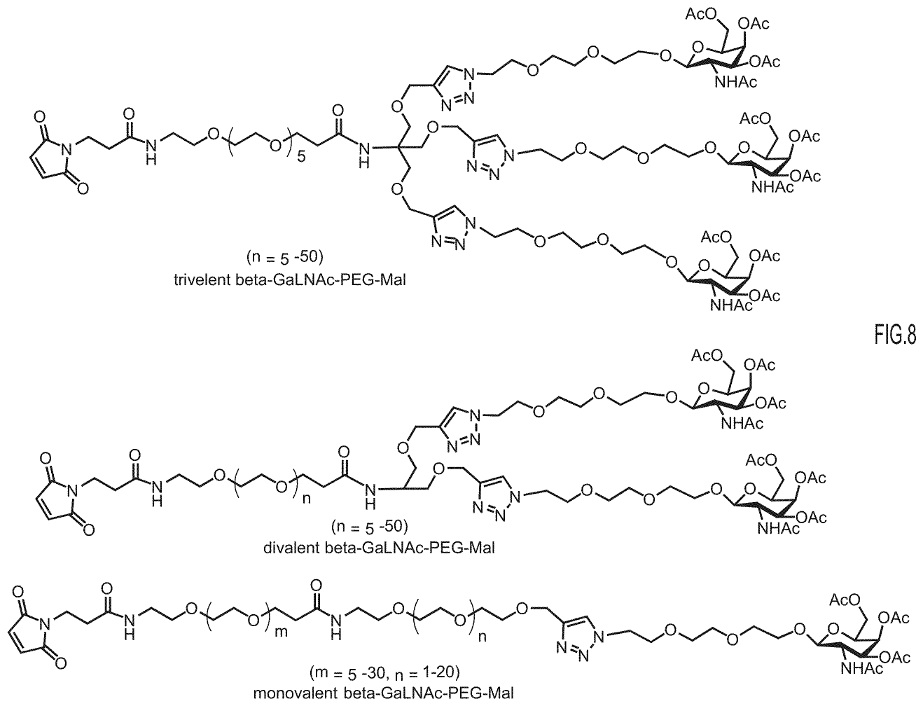

GalNAc-TGFβ1 in FIG. 3 m=0 was used in this study on human hepatocellular carcinoma HepG2 cells viability. Effect of treatment with Cell Death siRNA formulated with GalNAc-TGFβ1 (25 nM, 50 nM, 100 nM) along with control blank, non-silencing siRNA (NC, 100 nM), HKP (100 nM), liposome (25 nM, 50 nM and 100 nM, respectively), and liposome-GalNAc-TGFβ1 (25 nM, 50 nM, 100 nM, respectively). A mixture of GalNAc-TGFβ1 (25 nM, 50 nM, 100 nM, respectively), control blank, non-silencing siRNA (NC, 100 nM), HKP (100 nM), liposome (25 nM, 50 nM and 100 nM, respectively), liposome-GalNAc-TGFβ1 (25 nM, 50 nM, 100 nM, respectively) was incubated with cells in 100 μL OPTI-MEM medium. Transfection medium was replaced with 10% FBS/DMEM or EMEM after 6 h. At 72 h post-transfection, the number of viable cells was assessed along with a Real-Time Quantitative Reverse Transcription QRT-PCR assay to quantify the TGF-β1 mRNA relative expression. Values derived from untreated cells (Blank) were set as 100%. NC-non-silencing siRNA. See FIG. 8.

### Example 6. In Vivo Testing of GalNAc-TGFβ1 in Mouse Model

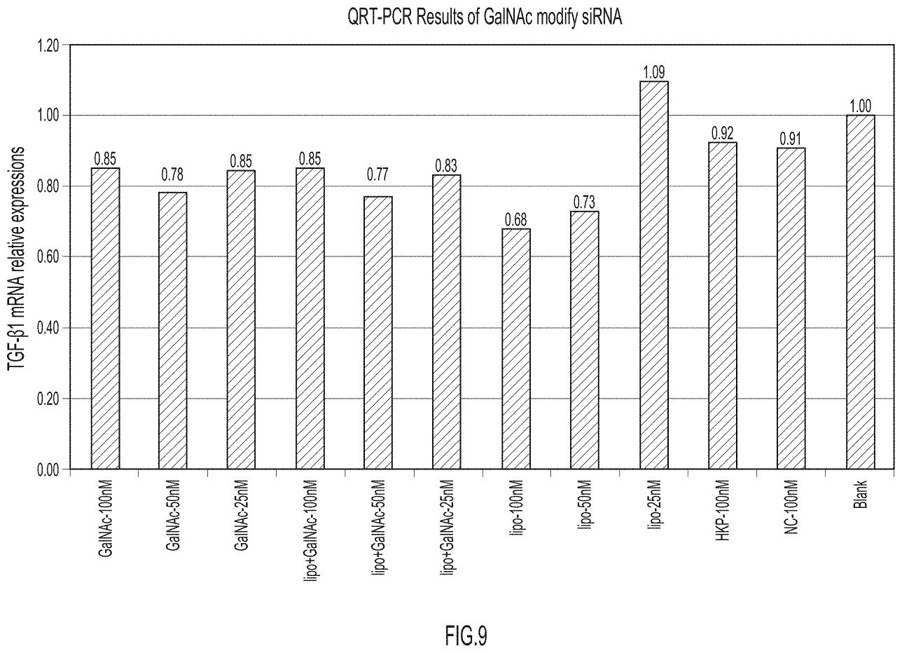

A group of 20 female mice at four weeks of age were divided into four groups. Dosages are siRNA per mouse and one injection dosages of 200 μg, 100 μg and 50 μg for GalNAc/siRNA-H to GalNAc/siRNA-L, PC (HKP/siRNA=4:1) is 40 μg. Each group was injected with the corresponding drug in the tail vein and injected once. Animals were sacrificed and the liver tissue was collected 24 hours after administration. The right lobe of liver tissue was homogenized for RNA extraction. qRT-PCR was then performed. Data shown are averages of 4 mice. *-P<0.05 V.S Blank, and **-P<0.01 V.S Blank. See FIG. 9. In the positive control (PC), the HKP/siRNA was delivered to the whole liver, however, the GalNAc-H, -M, -L are specific to the liver hepatocytes only. So the overall mRNA expression level is slightly higher in the GalNAc cases than the PC cases, but compares well with the blank (untreated). We observed the dose dependent effect of GalNAc-H, -M, and -L. Overall this strongly suggests that GalNAc had successfully delivered the siRNA and showed the silencing effect.

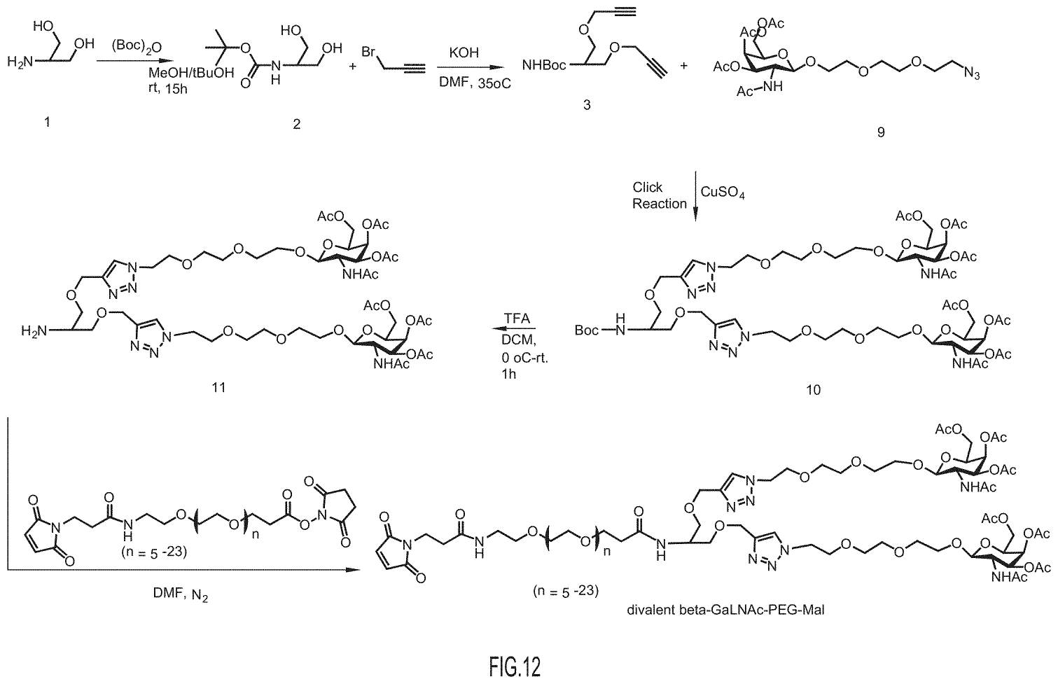

### Example 7. Preparation of Tripodal Compound 2 in FIG. 12.[7]

To a suspension of tris(hydroxymethyl)aminomethane (1) (10.0 g, 83.0 mmol) in t-BuOH (100 mL), a mixture of di-tert-butyl dicarbonate (23.4 g, 107.2 mmol) in MeOH:t-BuOH (160 mL, V/V=1:1) was added slowly under vigorous stirring and the reaction mixture was allowed to stir at room temperature for 15 h. After 15 h, the solvents were evaporated by using a rotavap to give a crude white solid which is recrystallized from ethyl acetate (300 mL) at room temperature. Vacuum filtration was used to collect the white needle shaped crystals which were washed by diethyl ether (100 mL). The solid was dried under vacuum for six hours to afford the pure product 2 as a white solid (17.0 g, 93%). The ¹H NMR data was in good agreement with the literature values. TLC (silica gel, Hexane:Ethyl acetate=5:1), ¹H NMR (400 MHz, DMSO-d6) δ: 5.77 (br s, 1H, NH), 4.50 (t, 3H, J=5.2 Hz, 3×OH), 3.50 (d, 6H, J=4.8 Hz, CH₂OH), 1.37 [s, 9H, 3×C(CH₃)₃] ppm.

### Example 8. Preparation of Tripodal Compound 3 in FIG. 12.[8]

To a solution of 4 (13.0 g, 58.7 mmol) in dry DMF, propargyl bromide (80 wt. % in toluene) (32.0 mL, 364.3 mmol) was added and the reaction mixture was stirred at 0° C. for 10 min. It was followed by the addition of finely powdered KOH (20.0 g, 364.3 mmol) in small portions. The entire reaction mixture was then stirred at room temperature for 40 h when the TLC (n hexane:EtOAc=5:1) showed the generation of a faster moving spot. To the resulting brown colored mixture, ethyl acetate was added and stirred for another 10 mins. Further, the entire reaction mixture was washed successively with H₂O (2×30 mL) and brine (25 mL). The organic ethyl acetate layer was collected, dried over anhydrous Na₂SO₄ and filtered. The solvents were then evaporated in vacuo. The crude material thus obtained was purified by flash chromatography using n-hexane:EtOAc as the eluent to yield the pure compound 5 (13.2 g, 67%) as a yellowish oil. 1H NMR (500 MHz, CDCl3) δ: 4.9 (br s, 1H, NH), 4.14 (d, 6H, 3×CH2CCH), 3.78 (s, 6H, CH2OH), 2.42 (t, 3H, 2.0 Hz, CCH), 1.42 (s, 1H, 3×C(CH3)3).

### Example 9. Preparation of Trivalent GalNAc-PEG6-Mal Ligand

A trivalent GalNAc-PEG6-Mal terminated with a maleimide was synthesized through 5 steps; compound 9 was coupled with compound 3 through “click” reaction to give compound 10. After the deprotection of Boc to give compound 11, compound 11 was then reacted with an N-hydroxysuccinimide group to yield the target compound trivalent GalNAc-PEG6-Mal ligand. See FIG. 12 for detail steps and example 1-3 for the characterization.

### Example 10. Preparation of Oligonucleotide-GalNAc Conjugate

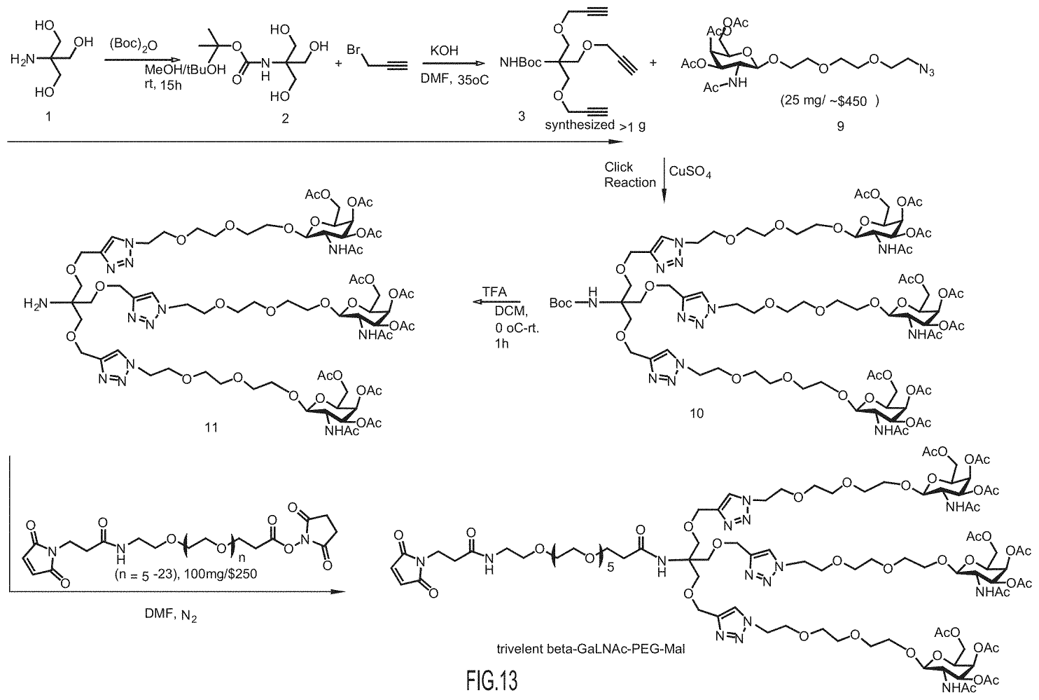

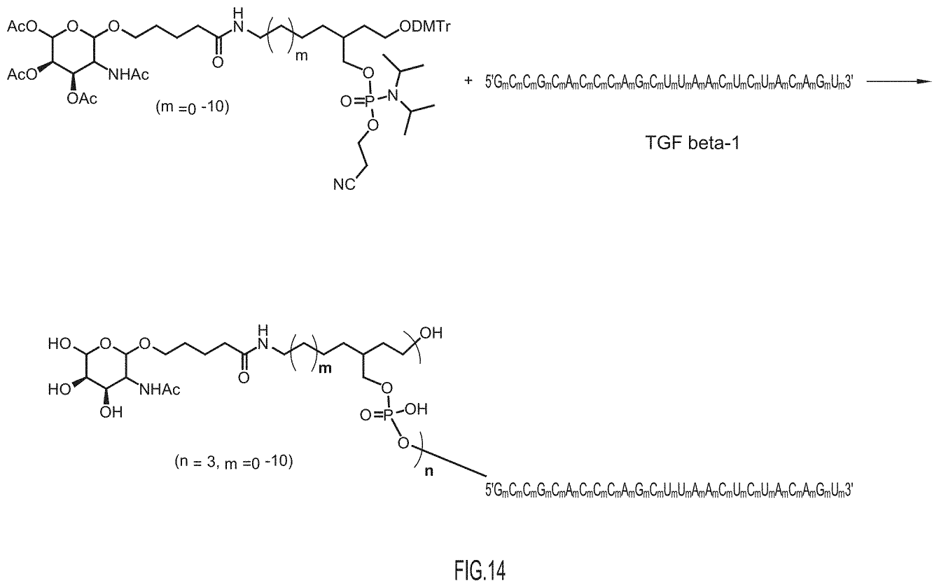

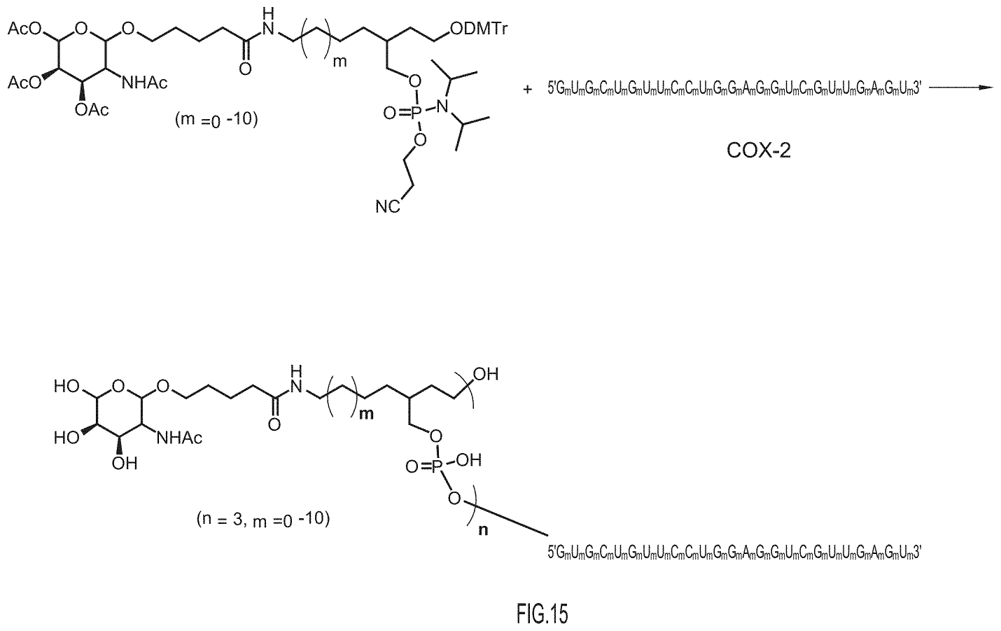

The oligonucleotides were prepared by the RNA ABI synthesizer with the designed sequence and functional moiety. See example in FIG. 15. The sense strand was modified by a thiol linker either via post-synthetic modification. Then the thiol modified sense strand was coupled with the trivalent beta-(GalNAc)3-PEG6-MAI in a phosphate buffer pH=7.5-9, the pure ligand conjugated oligonucleotide was obtained after the purification by gel-pak column or reverse C18 cartridge eluted in an acetonitrile and sodium acetate buffer. The siRNA duplex comprised two single oligonucleotides (both sense strand with ligand attached and antisense strand), in this case the 3′-sense strand was thiol modified by linker and GalNAc, then both strands were annealed by heating the mixture of the two single strands (sense:antisense ratio=1:1.05=nmol:nmol), at 90° C. for 5 min, then cooling it slowly at 1° C./min to room temperature. The resultant mixture was then stored at −20.0 overnight before use). Alternatively, the duplex was first annealed by a similar method using the sense strand with ligand modification and antisense strand. The annealed duplex was then used to couple with the trivalent beta-(GalNAc)3-PEG6-MAI in a phosphate buffer pH=7.5-9. The pure ligand-conjugated siRNA was obtained after removing the salts or use as is. See FIG. 12-15.

### 7. REFERENCES

[1]. Tatiparti K., Sau S., Kashaw S. K., Iyer A. K. (2017): siRNA Delivery Strategies: A comprehensive review of recent developments, Nanomaterials (Basel). 7(4), e77.
[2]. Yin Ren, Sangeeta N. Bhatia (2011): Targeted Delivery of Nucleicacids, US 900641562.
[3]. Kanasty R., DorKin R. J., Vegas A., Ander D. (2013): Delivery material for siRNA therapeutics, Nature Mater., 2013, 12, 967.
[4]. Barbara Bernardim, Maria J. Matos, Xhenti Ferhati, Ismael Compañón, Ana Guerreiro, Padma Akkapeddi, Antonio C. B. Burtoloso, Gonzalo Jiménez-Osés, Francisco Corzana & Gonçalo J. L. Bernardes, (2019): Efficient and irreversible antibody-cysteine bioconjugation using carbonylacrylic reagents, Nature Protocols, 14, 86-99.
[5]. Craig S. McKay, M. G. Finn, (2014) Click Chemistry in Complex Mixtures: Bioorthogonal Bioconjugation, Chemistry & Biology, 21, 1075-1101.
[6]. Lu P. Y., Xie F. Y. and Woodle M., (2003): SiRNA-Mediated Antitumorigenesis for Drug Target Validation and Therapeutics. Current Opinion in Molecular Therapeutics, 5, 225-234.
[7]. Soo Jung Son A, Margaret A. Brimble A D, Sunghyun Yang A, Paul W. R. Harris A, Tom Reddingius A, Benjamin W. Muir B, Oliver E. Hutt B, Lynne Waddington B, Jian Guan C and G. Paul Savage, (2013): Synthesis and Self-Assembly of a Peptide Amphiphile as a Drug Delivery Vehicle. Aust. J. Chem. 66, 23-29.
[8]. Das R., Mukhopadhyay B., (2016) Use of ‘click chemistry’ for the synthesis of carbohydrate-porphyrin dendrimers and their multivalent approach toward lectin sensing, Tetrahedron Letters, 57, 1775-1781.

All publications identified herein, including issued patents and published patent applications, and all database entries identified by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied without departing from the basic principles of the invention.

1. A chemical construct comprising the formula:
A-B—[—C-D]ₙ
wherein A comprises a first linker (linker 1), B comprises a bridge, C comprises a second linker (linker 2), D comprises a targeting ligand, and n is an integer from 1-4, wherein linker 1 and linker 2 can be the same or different, and wherein linker 1 comprises a linear polyethylene glycol as shown in the first structure below, wherein n1 is an integer between 1-50, or linker 1 comprises a poly(L-lactide) as shown in the second structure below, wherein n2 is an integer from 1-70, and wherein Z (shown in structures below) is a functional group, such as thiol or carboxylic acid, which will react with a maleimide or an amine to conjugate covalently with the bridge.

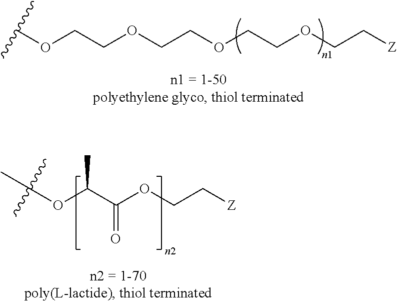

2. (canceled)

3. The construct of claim 1, wherein a chemical structure comprising linker 2 and 1-3 of the targeting ligands is attached to the bridge, wherein the chemical structure comprises one of the following structures: 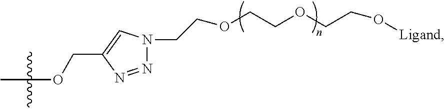
wherein n is 1, 2, or 3 and is connected to the bridge through a 1, 5-triazol ring with an OCH₂ unit; or 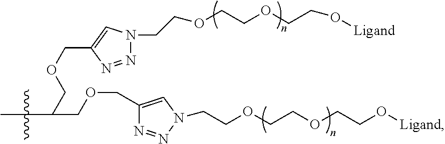
wherein n is 1, 2, or 3 and is connected to the bridge through a 1, 5-triazol ring with an CH₂OCH₂ unit; or 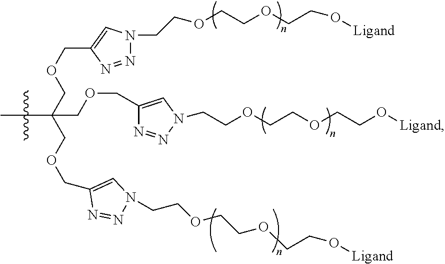
wherein n is 1, 2, or 3 and is connected to the bridge through a 1, 5-triazol ring with an CH₂OCH₂ unit.

4. The construct of claim 1, wherein linker 1 comprises a linear aliphatic chain conjugated by an internal amide bond and linker 2 and the bridge have been replaced with a phosphate linkage, as shown in the following structure: 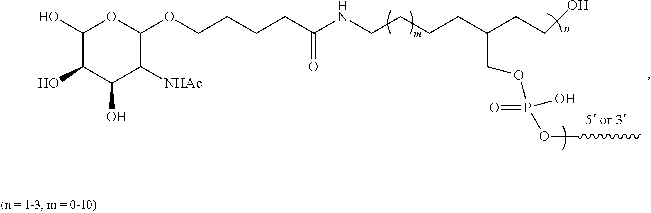
wherein m is 0-10 and n is 1-3.

5. The construct of claim 1, wherein the bridge comprises a chemical structure connecting linker 1 and linker 2, wherein the chemical structure is a linear structure —CH₂OCH₂—, a single-branched structure 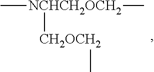
or double-branched tripodal structure 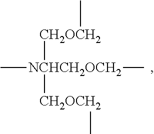
wherein the bridge is directly connected to the triazol ring of linker 2, and wherein the N terminal side of the bridge is linked to a short peg group terminated with a maleimide functional group or any other functional group that couples with linker 1 and
wherein the bridge is a linear structure with the formula 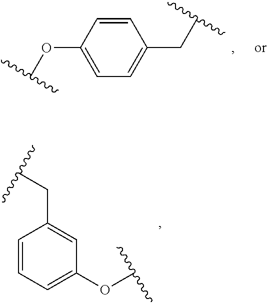 [SMILES: *Cc1ccc(O*)cc1 ; *Cc1cccc(O*)c1]
which allows only one chemical construct comprising linker 2 and a targeting ligand to be conjugated at the para-O position, or a branched structure with the formula 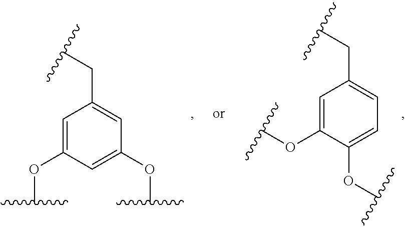 [SMILES: *Cc1cc(O*)cc(O*)c1 ; *Cc1ccc(O*)c(O*)c1]
which allows two chemical constructs comprising linker 2 and a targeting ligand to be conjugated at the two-O positions, or a tripodal structure with the formula 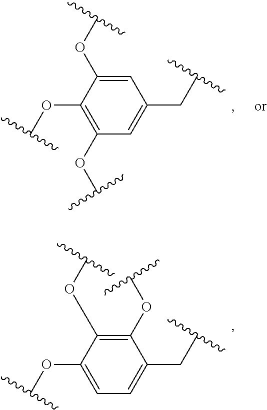 [SMILES: *Cc1cc(O*)c(O*)c(O*)c1 ; *Cc1ccc(O*)c(O*)c1O*]
which allows three chemical constructs comprising linker 2 and a targeting ligand to be conjugated at the three of three-O positions, wherein the CH₂ side of the bridge is linked to a short peg group terminated with a maleimide functional group or any other functional group that couples with linker 1.

6. (canceled)

7. The construct of claim 1, wherein linker 1 has a sub-chemical group Z comprising a thiol-maleimide bond as shown: 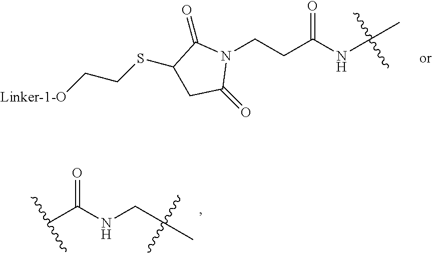
or any other pair of the conjugation chemistries listed below: 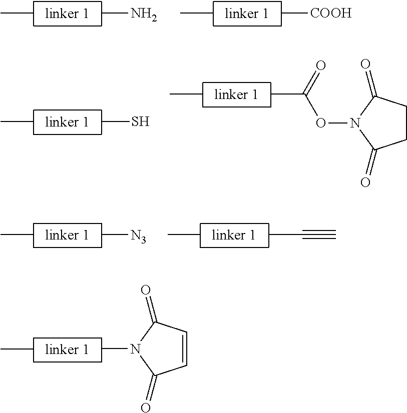
and wherein Z comprises a docking site that chemically attaches linker 1 and the bridge.

8. (canceled)

9. The construct of claim 1, wherein the targeting ligand is selected from the group consisting of N-acetyl-galactosamine (GalNAc), galactose, galactosamie, N-formal-galactosoamine, N-propionyl-galactosamine, and N-butanoylgalactosamine.

10. (canceled)

11. The construct of claim 1, wherein n is 2, 3 or 4.

12-13. (canceled)

14. The construct of claim 1, wherein linker 1 is attached to a therapeutic molecule selected from the group consisting of an expression-inhibiting oligonucleotide a therapeutic peptide, an antibody with therapeutic efficacy, and a small molecule with therapeutic efficacy.

15-17. (canceled)

18. The construct of claim 1, wherein the construct is covalently connected to an siRNA molecule at the 3′ position or the 5′ position through linker 1 as shown below, x=O or S, y=O or S:

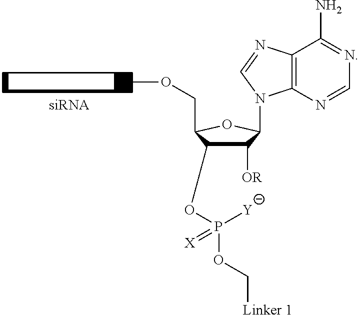

19. The construct of claim 14, wherein: the peptide comprises cyclic(c) RGD, APRPG (SEQ ID NO: 25), NGR, F3 peptide, CGKRK (SEQ ID NO: 26), LyP-1, iRGD (CRGDRCPDC) (SEQ ID NO: 27), iNGR, T7 peptide (HAIYPRH) (SEQ ID NO: 28), MMP2-cleavable octapeptide (GPLGIAGQ) (SEQ ID NO: 29), CP15 (VHLGYAT) (SEQ ID NO: 30), FSH (FSH-β, 33-53 amino acids, YTRDLVKDPARPKIQKTCTF) (SEQ ID NO: 31), LHRH (QHTSYkcLRP), gastrin-releasing peptides (GRPs) (CGGNHWAVGHLM) (SEQ ID NO: 32), RVG (YTWMPENPRPGTPCDIFTNSRGKRASNG) (SEQ ID NO: 33), FMDV20 peptide sequence (NAVPNLRGDLQVLAQKVART) (SEQ ID NO: 34), or GLP; the antibody for therapeutic use comprises IgM, IgD, IgG, IgA, IgE, or antibody fragments F(ab′)2, Fab, Fab′, or Fv; or the small molecule with therapeutic efficacy comprises gemcitabine, folic acid, cisplatin, oxaliplatin, carboplatin, doxorubicin, or paclitaxel.

20-21. (canceled)

22. A pharmaceutical composition comprising the construct of claim 14 and a pharmaceutically acceptable carrier wherein the pharmaceutically acceptable carrier comprises water and one or more of the following salts or buffers: potassium phosphate monobasic anhydrous NF, sodium chloride USP, sodium phosphate dibasic heptahydrate USP, and Phosphate Buffered Saline (PBS).

23. (canceled)

24. A method of delivering a therapeutic molecule to a human cell comprising delivering the construct of claim 14 to the cell in vivo.

25-28. (canceled)

29. The method of claim 24, wherein the cancer is selected from the group consisting of liver cancer, cholangiocarcinoma (CCA), colon cancer, pancreatic cancer, lung cancer, bladder cancer, ovarian cancer, head and neck cancer, esophageal cancer, brain cancer, and skin cancers, including melanoma and non-melanoma skin cancers.

30-36. (canceled)

37. The method of claim 24, wherein the therapeutic molecule comprises an siRNA molecule and the cell comprises a hepatocyte.

38. The method of claim 14, wherein the therapeutic molecule is delivered to a human for treating a disease selected from the group consisting of hepatitis, fibrosis, and primary sclerosing cholangitis (PSC).

39. The method of claim 38, wherein the therapeutic molecule comprises an siRNA.

40-41. (canceled)

42. A method of synthesizing the construct of claim 14, wherein the therapeutic molecule is an siRNA molecule, comprising the steps of:
conjugating the sense strand of the siRNA molecule to a functionalized linker-1 at the 5′ or 3′ site of the siRNA molecule through formation of a phosphate bond;
connecting number of one, (two or three) of the targeting ligand-linker 2 construct to the center linear linker, (dipodal, or tripodal bridge) site, wherein the other end of the bridge is conjugated with a short PEG group with a maleimide functional group at the end, which is used to conjugate linker 1 to the bridge;
conjugating the siRNA-linker-1 construct with the linker-2-targeting ligand construct through a thiol/maleimide reaction to provide a construct of the sense strand of the siRNA molecule with the one to three targeting ligand molecules; and
mixing the sense strand-targeting ligand construct with the antisense strand of the siRNA molecule to form the duplex siRNA with the one to three targeting ligands.

43. A method of synthesizing the construct of claim 14, wherein the therapeutic molecule is an antibody or a peptide, comprising the steps of:
conjugating the antibody (or peptide) molecule to a functionalized linker-1 (azido, maleimide, amine) at the alkyne, thiol, NHS functionalized site of the antibody (or peptide) molecule through formation of a triazole ring, a thiol-carbon bond, or an amide bond;
connecting number of one, (two or three) of the targeting ligand-linker 2 construct to the center linear linker, (dipodal, or tripodal bridge) site, wherein the other end of the bridge is conjugated with a short PEG group with a maleimide functional group at the end, which is used to conjugate linker 1 to the bridge;
conjugating the antibody (or peptide)-linker-1 construct with the linker-2-targeting ligand construct through a thiol/maleimide reaction to provide a construct of the antibody (or peptide) with targeting ligand.

44. A method of synthesizing the construct of claim 18, comprising the steps of:
connecting one, two or three of the targeting ligand-linker 2 construct to the center linear linker, (dipodal, or tripodal bridge) site, wherein the other end of the bridge is conjugated with a short PEG group with a maleimide functional group at the other end, which is used to conjugate linker 1 to the bridge;
reacting linker 1, such as PEG or poly(L-lactide) containing a thiol group moiety, with the terminal maleimide on the bridge-linker 2-GalNAc moiety to form a S—C covalent bond;
conjugating the ligand-linker 2-linker 1 construct to the 5′ or 3′ end of the sense strand of the siRNA molecule through a phosphate bond between the phosphonamidite group and the hydroxyl group; and
mixing the sense strand-GalNAc construct with the antisense strand of the siRNA molecule to form the siRNA duplex with the one to three GalNAc ligands.

45. A construct for delivering an oligonucleotide to a human hepatocyte, comprising the structure:
O-A-B—[—C-D]ₙ
wherein O comprises an oligonucleotide, A comprises a first linker (linker 1), B comprises a bridge, C comprises a second linker (linker 2), D comprises a targeting ligand, and n is an integer from 1-4 wherein the oligonucleotide comprises an siRNA that is between 10-27 nucleotides long and wherein the siRNA optionally is fully or partially chemically modified at a 2′ position or a phosphorothioate bond linkage.

46-60. (canceled)


SEQUENCE LISTING (37 sequences)

SEQ ID NO: 1 - RNA, 25 nt, Artificial Sequence
  Description of Artificial Sequence Synthetic oligonucleotide
ggauccacgagcccaagggcuacca

SEQ ID NO: 2 - RNA, 25 nt, Artificial Sequence
  Description of Artificial Sequence Synthetic oligonucleotide
ugguagcccuugggcucguggaucc

SEQ ID NO: 3 - RNA, 25 nt, Artificial Sequence
  Description of Artificial Sequence Synthetic oligonucleotide
cccaagggcuaccaugccaacuucu

SEQ ID NO: 4 - RNA, 25 nt, Artificial Sequence
  Description of Artificial Sequence Synthetic oligonucleotide
agaaguuggcaugguagcccuuggg

SEQ ID NO: 5 - RNA, 25 nt, Artificial Sequence
  Description of Artificial Sequence Synthetic oligonucleotide
gagcccaagggcuaccaugccaacu

SEQ ID NO: 6 - RNA, 25 nt, Artificial Sequence
  Description of Artificial Sequence Synthetic oligonucleotide
aguuggcaugguagcccuugggcuc

SEQ ID NO: 7 - RNA, 25 nt, Artificial Sequence
  Description of Artificial Sequence Synthetic oligonucleotide
gccgcacccagcuuaacucuacagu

SEQ ID NO: 8 - RNA, 25 nt, Artificial Sequence
  Description of Artificial Sequence Synthetic oligonucleotide
acuguagaguuaagcugggugcggc

SEQ ID NO: 9 - RNA, 25 nt, Artificial Sequence
  Description of Artificial Sequence Synthetic oligonucleotide
gccgcacccagcuuaacucuacagu

SEQ ID NO: 10 - RNA, 25 nt, Artificial Sequence
  Description of Artificial Sequence Synthetic oligonucleotide
acuguagaguuaagcugggugcggc

SEQ ID NO: 11 - RNA, 25 nt, Artificial Sequence
  Description of Artificial Sequence Synthetic oligonucleotide
gauccacgagcccaagggcuaccau

SEQ ID NO: 12 - RNA, 25 nt, Artificial Sequence
  Description of Artificial Sequence Synthetic oligonucleotide
augguagcccuugggcucguggauc

SEQ ID NO: 13 - RNA, 25 nt, Artificial Sequence
  Description of Artificial Sequence Synthetic oligonucleotide
cacgagcccaagggcuaccaugcca

SEQ ID NO: 14 - RNA, 25 nt, Artificial Sequence
  Description of Artificial Sequence Synthetic oligonucleotide
uggcaugguagcccuugggcucgug

SEQ ID NO: 15 - RNA, 25 nt, Artificial Sequence
  Description of Artificial Sequence Synthetic oligonucleotide
gaggucacccgcgugcuaauggugg

SEQ ID NO: 16 - RNA, 25 nt, Artificial Sequence
  Description of Artificial Sequence Synthetic oligonucleotide
ccaccauuagcacgcgggugaccuc

SEQ ID NO: 17 - RNA, 25 nt, Artificial Sequence
  Description of Artificial Sequence Synthetic oligonucleotide
guacaacagcacccgcgaccgggug

SEQ ID NO: 18 - RNA, 25 nt, Artificial Sequence
  Description of Artificial Sequence Synthetic oligonucleotide
cacccggucgcgggugcucuucuac

SEQ ID NO: 19 - RNA, 25 nt, Artificial Sequence
  Description of Artificial Sequence Synthetic oligonucleotide
guggauccacgagcccaagggcuac

SEQ ID NO: 20 - RNA, 25 nt, Artificial Sequence
  Description of Artificial Sequence Synthetic oligonucleotide
guagcccuugggcucguggauccag

SEQ ID NO: 21 - RNA, 25 nt, Artificial Sequence
  Description of Artificial Sequence Synthetic oligonucleotide
gugcuguuccuggaggucguugagu

SEQ ID NO: 22 - RNA, 25 nt, Artificial Sequence
  Description of Artificial Sequence Synthetic oligonucleotide
acucaacgaccuccaggaacagcac

SEQ ID NO: 23 - RNA, 25 nt, Artificial Sequence
  Description of Artificial Sequence Synthetic oligonucleotide
gugcuguuccuggaggucguugagu

SEQ ID NO: 24 - RNA, 25 nt, Artificial Sequence
  Description of Artificial Sequence Synthetic oligonucleotide
acucaacgaccuccaggaacagcac

SEQ ID NO: 25 - protein, 5 aa, Artificial Sequence
  Description of Artificial Sequence Synthetic peptide
APRPG

SEQ ID NO: 26 - protein, 5 aa, Artificial Sequence
  Description of Artificial Sequence Synthetic peptide
CGKRK

SEQ ID NO: 27 - protein, 9 aa, Artificial Sequence
  Description of Artificial Sequence Synthetic peptide
CRGDRCPDC

SEQ ID NO: 28 - protein, 7 aa, Artificial Sequence
  Description of Artificial Sequence Synthetic peptide
HAIYPRH

SEQ ID NO: 29 - protein, 8 aa, Artificial Sequence
  Description of Artificial Sequence Synthetic peptide
GPLGIAGQ

SEQ ID NO: 30 - protein, 7 aa, Artificial Sequence
  Description of Artificial Sequence Synthetic peptide
VHLGYAT

SEQ ID NO: 31 - protein, 20 aa, Artificial Sequence
  Description of Artificial Sequence Synthetic peptide
YTRDLVKDPARPKIQKTCTF

SEQ ID NO: 32 - protein, 12 aa, Artificial Sequence
  Description of Artificial Sequence Synthetic peptide
CGGNHWAVGHLM

SEQ ID NO: 33 - protein, 28 aa, Artificial Sequence
  Description of Artificial Sequence Synthetic peptide
YTWMPENPRPGTPCDIFTNSRGKRASNG

SEQ ID NO: 34 - protein, 20 aa, Artificial Sequence
  Description of Artificial Sequence Synthetic peptide
NAVPNLRGDLQVLAQKVART

SEQ ID NO: 35 - protein, 4 aa, Artificial Sequence
  Description of Artificial Sequence Synthetic peptide
HHHK

SEQ ID NO: 36 - protein, 5 aa, Artificial Sequence
  Description of Artificial Sequence Synthetic peptide
HHHHK

SEQ ID NO: 37 - protein, 20 aa, Artificial Sequence
  Description of Artificial Sequence Synthetic peptide
  MISC_FEATURE (1)..(20): This sequence may encompass 1-5 “His His His Lys” repeating units
HHHKHHHKHHHKHHHKHHHK